(19) United States
(12) Patent Application Publication
(10) Pub. No.: US 2015/0057572 A1
(43) Pub. Date: Feb. 26, 2015

(54) BIOPSY AND SUTURELESS DEVICE

(71) Applicants: Jose Arturo Mendez-Coll, San Juan, PR (US); Felix Omar Rivera Ortiz, Caguas, PR (US); Yahveh Comas Torres, San Juan, PR (US)

(72) Inventors: Jose Arturo Mendez-Coll, San Juan, PR (US); Felix Omar Rivera Ortiz, Caguas, PR (US); Yahveh Comas Torres, San Juan, PR (US)

(21) Appl. No.: 13/975,255

(22) Filed: Aug. 23, 2013

(51) Int. Cl. A61B 17/064; A61B 10/02

(52) U.S. Cl. CPC A61B 17/0644; A61B 10/0266; A61B 2010/0208
USPC 600/567

(57) ABSTRACT

A dermal punch device for automatically extracting a sample of tissue of a predetermined size and shape from a body comprising a retractable cutter and a sutureless biopsy closure mechanism that includes a wound closure fastener member adapted to be disposed over a biopsy region after the performance of the biopsy, wherein wound closure fastener member is automatically applied without the need of several instruments to seal the wound. The wound closure fastener member is dispensed by a sutureless biopsy closure dispenser located at the same distal end of the biopsy punch device surrounding the biopsy punch cutter assembly avoiding the need of separates instruments, reducing the wound closing steps and surgical procedure time.

### RELATED APPLICATIONS

This application is related to Non-Provisional U.S. patent application Ser. No. 13/089,056 filed on Apr. 18, 2011.

### STATEMENT REGARDING FEDERALLY SPONSORED RESEARCH AND DEVELOPMENT

N/A

### BACKGROUND OF THE INVENTION

1. Field of the Invention

This application relates generally to the field of extracting tissue samples from solid bodies, and more specifically to surgical instruments for extracting a biopsy or sample of tissue while providing a sutureless biopsy wound site closure.

2. Discussion of the Background

Annually thousands of persons and animals are tested for numerous skin problems such as abnormal skin growths and cancers, as well as skin eruptions. Surgical instruments, such as a dermal punch and others are used to obtain samples of skin lesions for diagnostic purposes. The procedure involves the insertion of a cutting surgical instrument into the patient's skin wherein the cutting surgical instrument comprises a cylindrical blade at the distal end of an inert plastic or metal rod. After abutting the cylindrical blade to the skin the cutting instrument is rotated so as to cut out and remove a plug of the tissue of interest. The tissue is then submitted to be analyzed by a pathologist in order to obtain a diagnosis.

For example in a skin punch biopsy, the skin surrounding the lesion is pulled taut, and the punch is firmly introduced into the lesion and rotated to obtain the tissue specimen. The punch must go deep enough to include an average of a 6 mm depth in order to include the lower dermis and subcutaneous fat. The plug is lifted with forceps or a needle. The specimen is placed in a properly labeled sterile container.

After the removal of the tissue, the traditional dermal punch biopsy usually leaves a circular wound opening which is then normally closed by a suture. Some of the problems associated with this technique include, but are not limited to, the use of multiple instruments in performing the suturing which typically requires at least a needle holder, scissors, suture material and forceps. Other problems are the potential for a needle stick injury and the increased cost of the suture and sterilization of the instruments used. Furthermore, there is also a need for more than one person to perform the procedure, for example a nurse has to prepare a sterile instrument tray, pass the instruments to the surgeon, place a bandage on the wound following the procedure and finally pick up and resterilize the instruments.

There is a need for a device that provides a streamlined procedure which does away with the time consuming pre and post operative phase, reduces the currently needed personnel, and instruments and provides an improved sutureless wound closure at the site of the biopsy wound. It should be relatively inexpensive, easy to apply, efficient and not require subsequent procedures.

### SUMMARY OF THE INVENTION

The present disclosure describes a biopsy punch device operated by a single operator, wherein the biopsy punch device comprises a mean to obtain the tissue sample while achieving sutureless closing of the biopsy wound site without a need of other instruments. Comparable to biopsy punch device disclose in patent application Ser. No. 13/089,056 filed on Apr. 18, 2011, herein included by reference, the present biopsy punch device cuts and extracts the tissue without lifting the device from the skin. After the tissue is removed the biopsy punch device achieves sutureless closing of the biopsy wound site without the need for additional instruments such as needles, suture material, forceps and scissors. The suture process is achieved by deploying a fastener member having resilient properties configured for this purpose and mechanically activated by the cutter assembly.

The first embodiment of the biopsy punch device comprises an elongated hollow cylinder with a distal end having an preloaded exposed cylindrical cutter which rotates during the incision procedure and is then retracted into the body of the elongated hollow cylinder while the cylindrical cutter assists with the grasping and removing of the tissue sample and; wherein said cylindrical cutter is coupled to a sutureless fastening mechanism. The sutureless fastening mechanism comprises a sutureless dispenser to dispense a biopsy closure fastener member. The sutureless biopsy closure dispenser, more particularly the trigger is activated by a means of a linear motion provided by the cutter assembly which assists to release at least a biopsy closure fastener member at the wound site which renders the biopsy wound site closed by approximating the opposing edges of the wound. The biopsy closure fastener member comprises at least two-pronged fastener having resilient properties, wherein the two-pronged fastener may expand to a distance at least equal to the diameter of the wound created by the cylindrical cutter and then contracts when released therein. The present biopsy punch device eliminates the need for multiple instruments and their handling, sutures and speeds-up the process.

It is another objective to provide a retractable biopsy punch actuated by an manual, wherein the biopsy punch cutter comprises a constant and uniform depth to provide a tissue specimen having a uniform thickness for accurate diagnosis.

It is a further objective to provide a biopsy punch device that is inexpensive to manufacture such that it can be made disposable.

It is a further objective to provide a biopsy punch cutter blade that is configured to assist with the removal of the tissue sample.

Another objective is to provide a mechanical structure that automatically activates the sutureless dispenser for suturing during the procedure without the need of several instruments.

Another objective is to minimize time and effort during the process of closing the wound site.

The preferred embodiment for a biopsy and sutureless device constructed pursuant to this application, both as to its configuration and its mode of operation will be best understood, and additional objects and advantages thereof will become apparent, by the following detailed description taken in conjunction with the accompanying drawings.

The applicant hereby asserts, that the disclosure of the present application may include more than one invention, and, in the event that there is more than one invention, that these inventions may be patentable and non-obvious one with respect to the other.

Furthermore, the purpose of the accompanying abstract is to enable the U.S. Patent and Trademark Office and the public generally, and especially the scientists, engineers, and practitioners in the art who are not familiar with patent or legal terms or phraseology, to determine quickly from a cursory inspection the nature and essence of the technical disclosure of the application. The abstract is neither intended to define the disclosure of the application, which is measured by the claims, nor is it intended to be limiting as to the scope of the disclosure in any way.

### BRIEF DESCRIPTION OF THE DRAWINGS

The accompanying drawings which are incorporated herein constitute part of the specifications and illustrate the preferred embodiment of a biopsy and sutureless device constructed pursuant to an example embodiment of the present invention.

FIG. 1 is a perspective view of an exemplary biopsy punch device in accordance with the principles of the present example embodiment of the present invention.

FIGS. 2A through 2B are perspective views of an exemplary biopsy punch device without housing in accordance with the principles of the present example embodiment of the present invention.

FIG. 3 is a perspective view of an exemplary biopsy punch device housing in accordance with the principles of the present example embodiment of the present invention.

FIG. 4 is a sectioned view of an exemplary biopsy punch device in accordance with the principles of the present example embodiment of the present invention.

FIG. 5 is a sectioned view of an exemplary biopsy punch device housing in accordance with the principles of the present example embodiment of the present invention.

FIG. 6 is a sectioned view of an exemplary biopsy punch device without housing in accordance with the principles of the present example embodiment of the present invention.

FIG. 7 is a perspective view of an exemplary cutter assembly in accordance with the principles of the present example embodiment of the present invention.

FIG. 8 is a perspective and exploded view of an exemplary biopsy cutter in accordance with the principles of the present example embodiment of the present invention.

FIG. 9 is a perspective sectional view of an exemplary biopsy cutter in accordance with the principles of the present example embodiment of the present invention.

FIG. 10 is a perspective view of an exemplary embodiment of the cutter assembly shaft in accordance with the principles of the present example embodiment of the present invention.

FIG. 11 is a perspective view of an exemplary embodiment of the cutter assembly shaft upper section in accordance with the principles of the present example embodiment of the present invention.

FIG. 12 is a perspective view of an exemplary embodiment of the cutter assembly shaft gearing in accordance with the principles of the present example embodiment of the present invention.

FIG. 13 is a perspective view of an exemplary embodiment of the cutter assembly shaft rotational pivot in accordance with the principles of the present example embodiment of the present invention.

FIG. 14 is a perspective view of an exemplary embodiment of the sutureless dispenser actuator surrounding the cutter assembly shaft in accordance with the principles of the present example embodiment of the present invention.

FIG. 15 is a perspective view of an exemplary embodiment of the sutureless dispenser actuator in accordance with the principles of the present example embodiment of the present invention.

FIG. 16 are alternative perspective view of an exemplary embodiment pointing out the hammer configuration of the sutureless dispenser actuator in accordance with the principles of the present example embodiment of the present invention.

FIGS. 17A and 17B are perspective views of an exemplary embodiment of the hammer configuration of the sutureless dispenser actuator in accordance with the principles of the present example embodiment of the present invention.

FIG. 18 is a perspective view of an exemplary embodiment of the sutureless dispenser actuator body in accordance with the principles of the present example embodiment of the present invention.

FIG. 19 is sectional view of an exemplary embodiment of the sutureless dispenser actuator body in accordance with the principles of the present example embodiment of the present invention.

FIGS. 20A-20C are side views of an exemplary embodiment of the biopsy punch device loading process in accordance with the principles of the present example embodiment of the present invention.

FIGS. 21A-21C are perspective view of an exemplary embodiment of the biopsy punch device loading process in accordance with the principles of the present example embodiment of the present invention.

FIG. 22 is perspective view of an exemplary embodiment of the interaction between the cutter and sutureless dispenser assembly at the compress stage in accordance with the principles of the present example embodiment of the present invention.

FIG. 23A-23B are perspective views of an exemplary embodiment of the interaction between the cutter and sutureless dispenser assembly at the decompress stage in accordance with the principles of the present example embodiment of the present invention.

FIG. 24 is a perspective view of an exemplary embodiment of the hammer at the suturing stage in accordance with the principles of the present example embodiment of the present invention.

FIG. 25A-25B is a perspective view of an exemplary embodiment of the sutureless fastener member in accordance with the principles of the present example embodiment of the present invention.

### DESCRIPTION OF THE PREFERRED EMBODIMENT

FIG. 1 shows an exemplary biopsy punch device in accordance with the principles of the present application. The first embodiment for a biopsy punch device 1 constructed in accordance with this application comprises an elongated hollow body housing comprising a grip zone 2a, wherein said housing 2 surrounds the sutureless dispenser assembly 3 and the cutter assembly 4. A rotational pivot is located and mechanically attached to the cutter assembly 4 with a distal end having an exposed cylindrical cutter 3.

FIGS. 2A through 2B are perspective views of an exemplary biopsy punch device without housing 2 in accordance with the principles of the present example embodiment of the present invention. FIG. 2A shows the portion of the sutureless dispenser actuator 3, wherein a housing stopper 31 protrudes from the sutureless dispenser actuator body. The housing stopper 31 avoids or prevents the unwanted displacement of the housing 2. Further a piston or hammer 7, is designed to be in close contact with the sutureless fastener member F. As shown in U.S. patent application Ser. No. 13/089,056 hereby included by reference, the hammer serves to punch the fastener into patience skin during the pushing action. The hammer 7 is mechanically coupled to a resilient member 6, such as spring. The resilient member 6 limits the displacement of the hammer 7. FIG. 2B represents opposite side of the sutureless dispenser actuator 3. The distal end 8 of the sutureless dispenser actuator 3 is configured to matches the hammer 7 body in such way that provides a path, aligns and limits the displacement of the hammer 7 during the reciprocal displacement and avoids or prevents that the hammer travels further than the distal end 8.

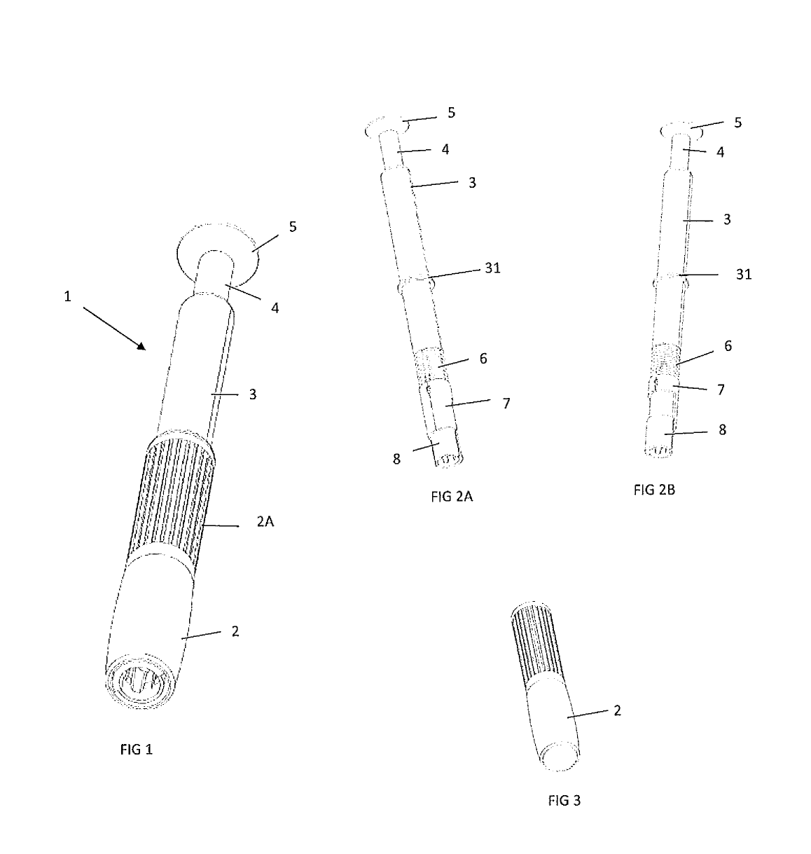

FIG. 3 is directed to the housing 2 which comprises a durable material which is not easily deformed when an user provides pressure over the housing 2 structure. Further the housing 2 comprises a grip zone 2a in order to assists the transmission of rotational movement over the biopsy punch device 1 by the user.

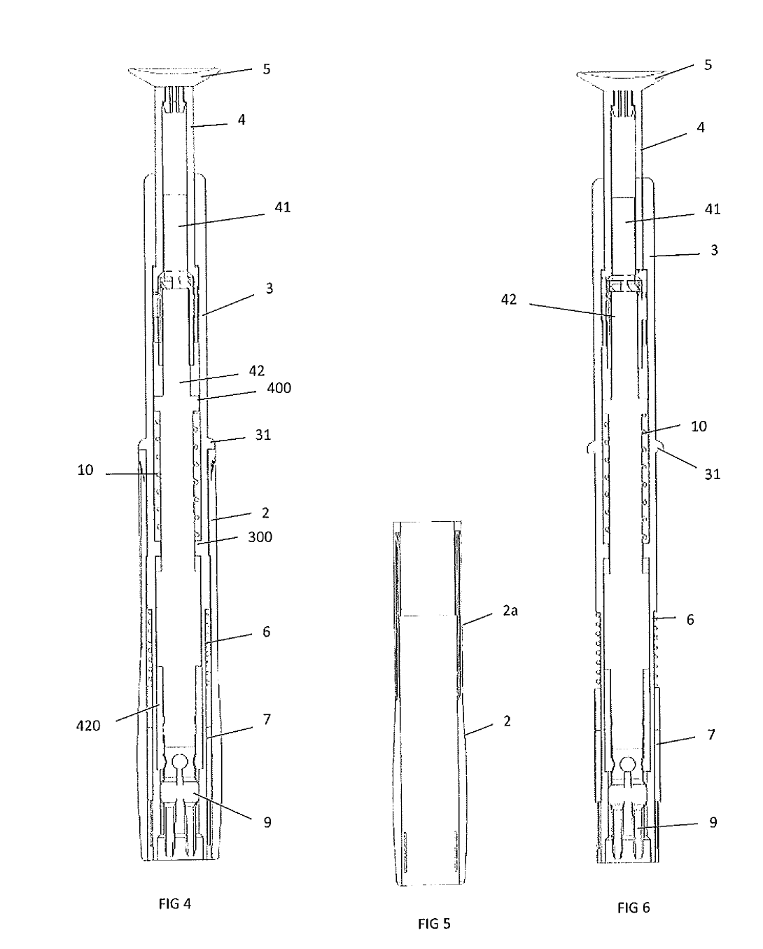

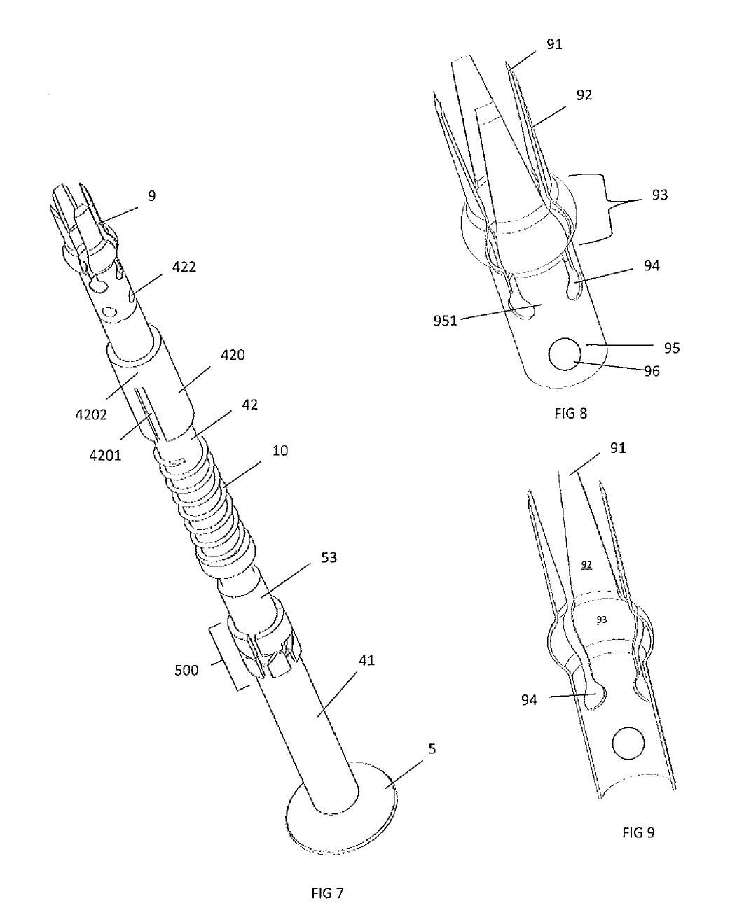

FIG. 4 shows the inner structure of the biopsy punch device 1, but more particularly the inner structure. FIG. 5 and FIG. 6 are directed to sectional views of the housing 2 and the punch device 1 without said housing 2. The exemplary embodiment shows a housing 2 surrounding the sutureless dispenser 3 and the cutter assembly 4. As mentioned before the hammer 7 and resilient member 6 surrounds the cutter shaft 42. Further is clearly showed how the housing 2 is in contact with the stopper 31. The cutter assembly 4, more particularly the upper shaft section 42 is surrounded by a second resilient material 10. The main purpose of the second resilient material 6 is to provide a linear displacement of the cutter shaft 4. The second resilient material 10 is located between the sutureless dispenser 3 inner protrusion 300 and cutter shaft stopper 400. The cutter 9 is located at a distal end of an upper shaft section 42. In addition is clearly shown that the rotational pivot 5 is mechanically coupled to the cutter assembly shaft 4. A portion a protrusion of said rotational pivot 5 extend inside the hollow body of the lower shaft section 41. The sutureless dispenser 3, has a hollow body with an inner surface configured to provide a several reduction in order to mechanically coupled some features such as resilient material 6 and particular shaped having a tapered zone 303 defining a circular contour toward the inner surface of the sutureless dispenser 3 structure in order to provide smooth contact with the cutter and assisting with the collapsing action of said cutter during the linear displacement.

FIG. 7 is directed to the cutter shaft assembly. The cutter shaft assembly comprises a cutter 9 located at an elongated body, wherein said elongated body 4 comprises a distal end of a upper shaft section 42, a lower section 41, a second resilient material 10, a gear assembling 9 and a rotational pivot 5.

The cutter 9, as shown in FIG. 8 and FIG. 9, comprises at a plurality of blades made of an inexpensive and durable metal and/or plastic materials such as stainless steel blade 92, a middle cutter body 93, tapered gap 94, a cutter base 95 and fixing means 96. The pluralities of blades are arranged in a substantially circular contour, wherein each blade is separated by the tapered gap 94. The tapered gap 94 is configured to comprise a shape that assists the bending action of each blade with respect to the base 95. For example, the reduce body portion 951 of the blade 92 closer to the base 95 is reduced in order to provide less resistance while the blade 92 is bend during the compression action of the middle cutter body 93. The tapered gap 94 extends from the blade through the middle cutter body 93 until reaching the cutter base 95 as show in FIG. 9.

Further the middle cutter body 93 is shaped to comprise an articulate configuration. The articulate configuration connects the blades 92 and the cutter base 95. The articulate configuration serves to promote a distal compression or displacement of the tips 91 of the blades 92 toward each other when the middle cutter body 93 contacts the dispenser reduced inner diameter or tapered zone 303 during the retraction of the cutter 9 inside the sutureless dispenser 3.

The tapered gap 94 allows the bending action of each blade 92 without the deforming, in order word while the compressing force is exerted at the middle cutter body 93 each blade tip 91 move toward the blades that is facing in such way that substantially circular contour diameter is reduced. While applying compressing force to the middle body 93 the tapered tap 94 or space between the blades 92 is reduced and simultaneously the blades tips 91 get closer. The space reduction results in a reduction of diameter at the space between tip blades 91 serves as a grasping action. Further a receiving section 96 is provided at a proximal end of the cutter 9. The attaching section 96 serves to hold the blade in position at the upper shaft section 42.

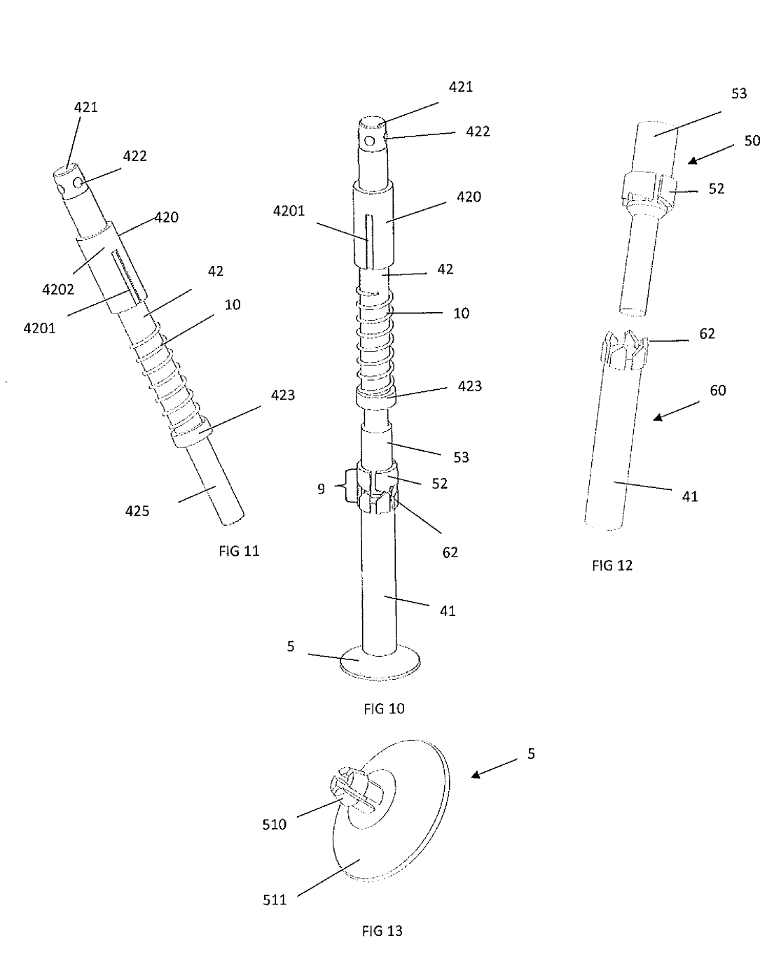

FIG. 10 is directed to the cutter shaft assembly without the cutter 9. The upper section or upper shaft end 42 comprises a distal end 421, a cutter holder 422, a dispenser trigger 420, a second resilient material stopper 400 and a proximal end 425, as shown in FIG. 11. The dispenser trigger 420 comprises at least a groove 4201 and solid section 4202.

FIG. 12 is directed to the cutter shaft gear assembling 500, wherein said assembling comprises a gear first end or follower 50 and a gear second end or push section 60. The cutter shaft gear assembly 500 assists to retract and extend the cutter shaft, more particularly retract and extend the cutter 9 away and inside the housing 2. In the preferred exemplary embodiment the cutter shaft gear assembly 500 is a cam follower mechanism comparable to the system used in ball points. The follower 50 comprises a transitional section 52, a first extension 53 and a second extension 54. The second gear end comprises a displacement actuator 62. The second extension 54 is located inside the lower shaft end 41 in such way displacement actuator contacts the transitional section 52. The geometry of the displacement actuator 62 and the transitional section 52 in combination with the spring 10 provides a linear displacement of the cutter 9. The cam mechanism 500 at least provide two stages; the compressing stage, wherein the cutter 9 extends away from the housing 2 and a decompress stage wherein said cutter 9 is pushed inside the sutureless dispenser 3. The combination of the first gear 50 and second gear 60 provides a linear displacement of the upper shaft 42, more particularly the cutter 9. It is important to understand that several gear mechanism 500 capable of retracting and extending the cutter 9 away and inside from the housing 2 can be used.

FIG. 13 is directed to the rotational pivot 5 comprising an extended attaching member 510 and wheel 511. As previously mentioned, the attaching member 510 is inserted and mechanically attached to rower shaft section 41. The rotational pivot 5 rotates independently form the biopsy punch device 1 by means of a attaching member 510 serving as a bearing, therefore the user can rotate the punch device by the housing 2, more particularly by means of the grip 2a, while applying vertical pressure on the rotational pivot 5 during the insertion and tissue removing process of a patient.

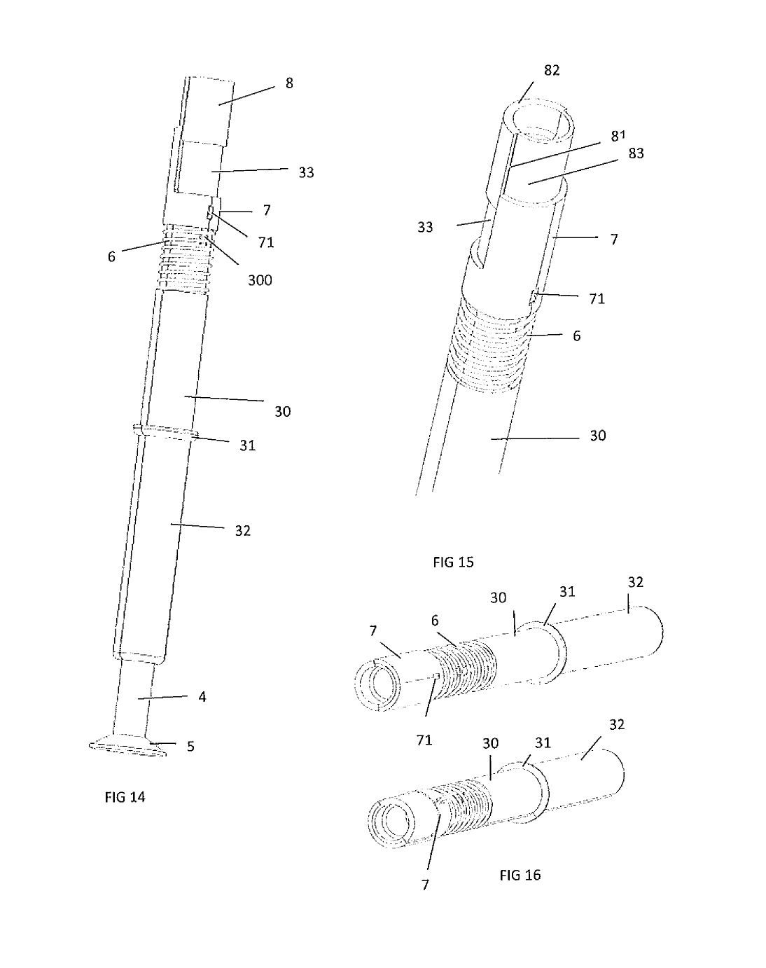

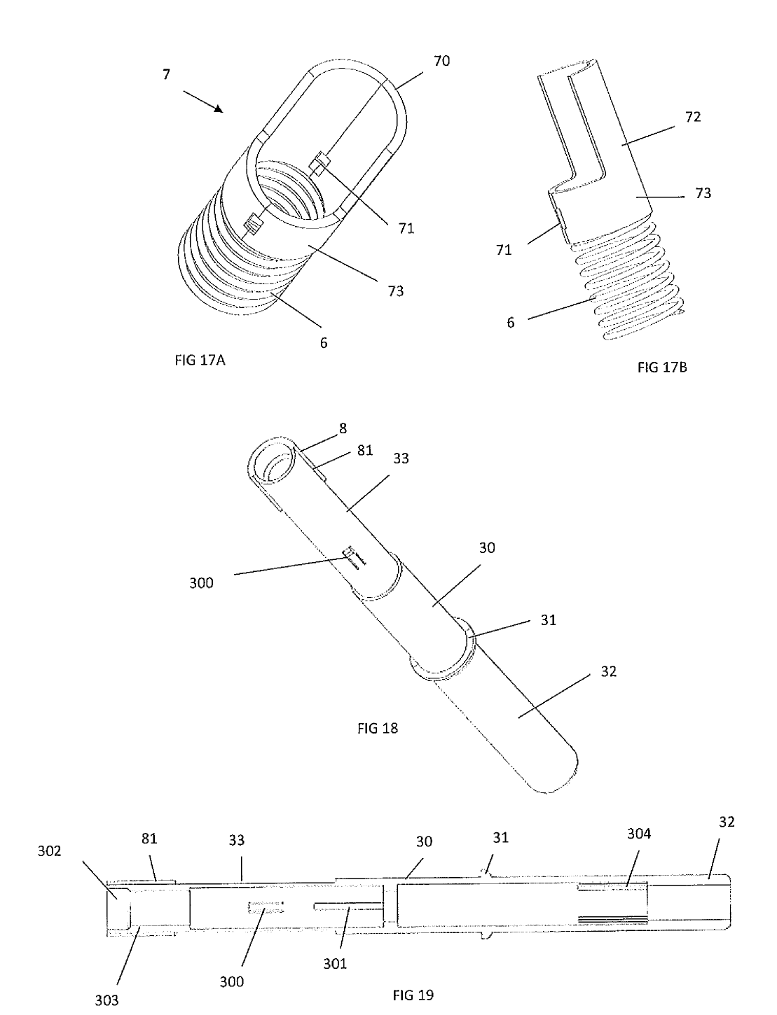

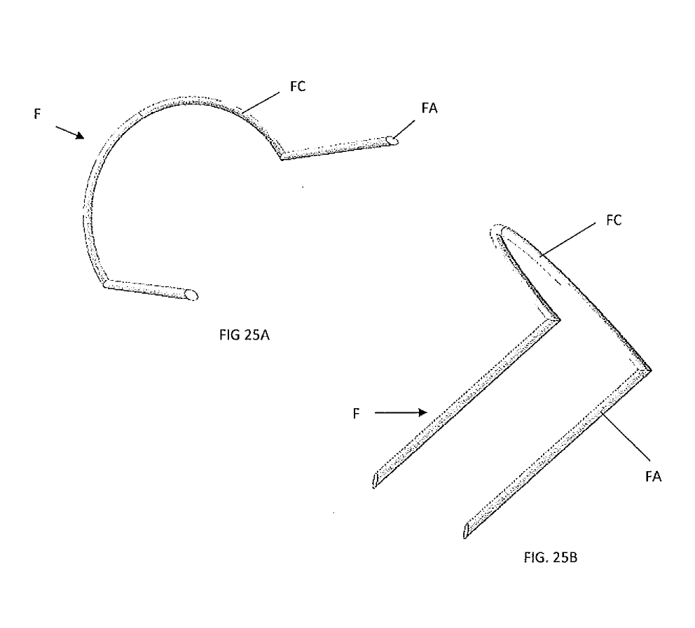

FIG. 15 through FIG. 21 are directed to the sutureless dispenser actuator 3. As shown in FIG. 14 through FIG. 16, the sutureless dispenser actuator 3 comprises a sutureless fastener F, a dispenser sleeve divided into an upper dispenser section 30 and a lower dispenser section 32, a stopper 31 and a actuator mechanism, wherein said actuator mechanism comprises a hammer 7 and a resilient material 6. The upper dispenser section 30 comprises a reduce diameter outer section 33 and a distal end protrusion 8. The distal end protrusion 8 comprises a reduce protrusion section 83, a solid protrusion section 82 and a travel surface 81. As mentioned before, the distal end protrusion 8 of the sutureless dispenser actuator 3 is configured to matches the hammer 7 body in such way that provides a path, aligns and limits the displacement of the hammer 7 during the reciprocal displacement and avoids or prevents that the hammer travels further than the distal end 8. A portion of the hammer 7 travels on top of travel surface 81 of the distal end protrusion 8 until contacting the a solid protrusion section 82.

The hammer 7, as shown in FIG. 17A through 17B, comprises a hollow structure with a fastener contacting area 70, a sectional area 72, a solid hammer area 73 and a recess 71. The sectional area 72 is configured to match distal end protrusion 8 in such way that contacts the solid protrusion section 82 and stop moving over the traveling surface 81.

Further, the recess 71 serves as a receiving section for holding element 300. While the resilient material 6 is compressed the holding element 300 is inserted inside the recess 71 retaining the resilient material 6 in a compressed position. The holding element 300 is released or moved from the recess 71 by means of the dispenser trigger 420 when the cutter shaft 4 is retracted. As mentioned before the sutureless dispenser actuator 3 surrounds the cutter shaft assembly. The inner surface of the sutureless dispenser actuator 3 is configured to provide support to several features of the cutter shaft. In the instant case the sutureless dispenser actuator 3 comprises a dispenser reduced inner diameter or tapered zone 303. The tapered zone 303 serves to promote a distal compression or displacement of the tips 91 of the blades 92 toward each other by contacting the middle cutter body 93 during the retraction of the cutter 9 inside the sutureless dispenser 3.

Further, as shown in FIG. 19, the sutureless dispenser 3 comprises a protrusion 301 which fits inside the groove 4201 in order to avoid rotational movement of the upper shaft 42. The dispenser trigger 420 is configured to rests inside inner surface of the sutureless dispenser 3 wherein the solid section 4202 contacts the holding element 300 in such way that pushes away providing the insertion of the holding element 300 inside the recess 71. The contact of the solid section 4202 with the holding element 300 is provided during the stage where the spring 10 is compressed and the cutter is extended away from the housing 2. Once the stage changes to retract the cutter 9 said solid section 4202 stops or discontinues the contact with the holding element 300. Therefore the holding member 300 is not pushed inside the recess 71 resulting in the release and linear movement of the hammer 7 due to the decompression of the resilient member 6.

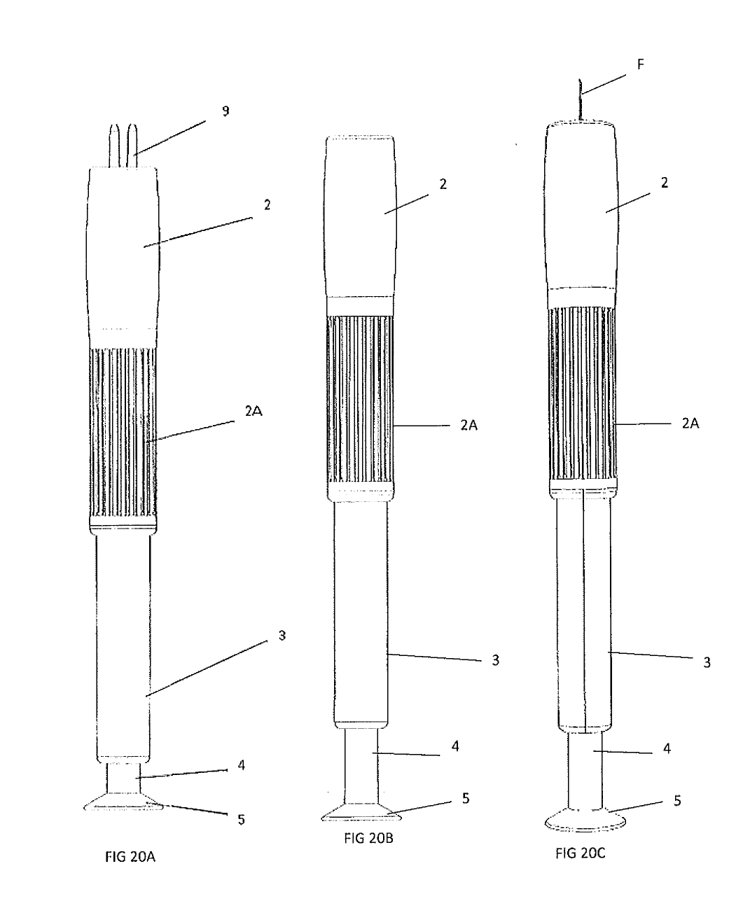

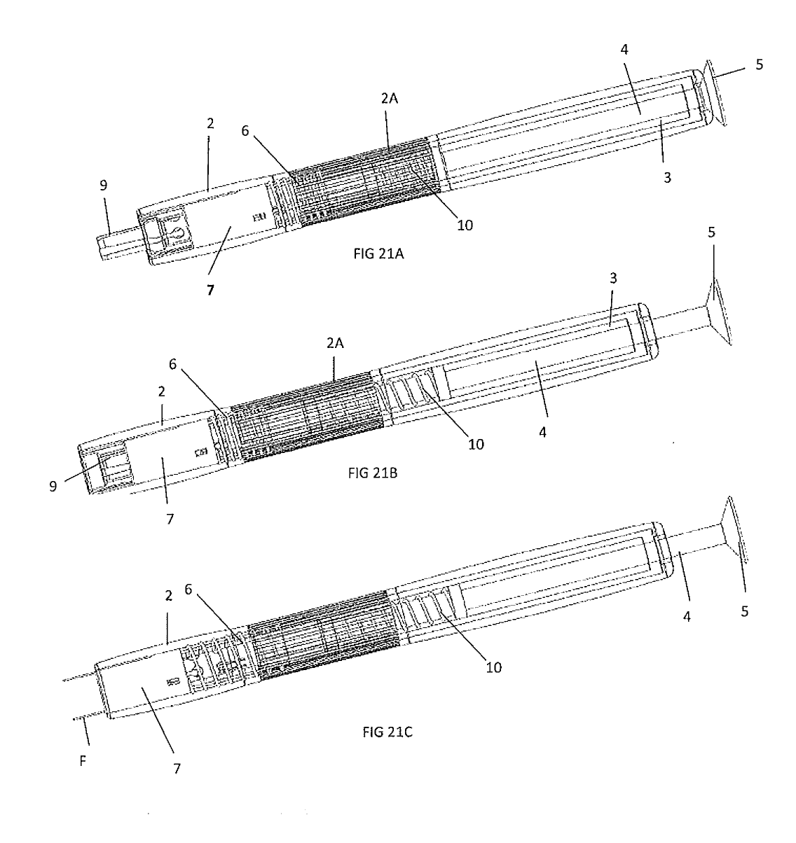

FIG. 20A through 24 are directed to the different stages. FIG. 20A discloses the compressing stage, wherein the cutter 9 extends away from the housing 2. During the compressing stage the resilient member 10 is compressed, as showed in FIG. 21A, and the second resilient member 6 is also compressed holding the hammer 7. This stage is also known as loading stage. Further the next stage is the decompress stage, as shown FIG. 20B, wherein said cutter 9 is pushed inside the sutureless dispenser 3. The decompress stage is accomplish by means of the shaft gear 500. By pressing the first gear 50 the second gear 60 rotates and generates a linear motion of the cutter 9. The resilient member 10 is decompressed and pushes the cutter 9 inside the housing 2. Once the cutter 9 get inside of the sutureless dispenser the contact between the solid section 4202 stops or discontinues with the holding element 300. This action results in the last and final stage, the suturing stage.

As mentioned the holding member 300 is not pushed inside the recess 71 resulting in the release and linear movement of the hammer 7 due to the decompression of the resilient member 6. The hammer 7 pushes the fastener F towards the patient body, as shown in FIG. 20 C and FIG. 21C.

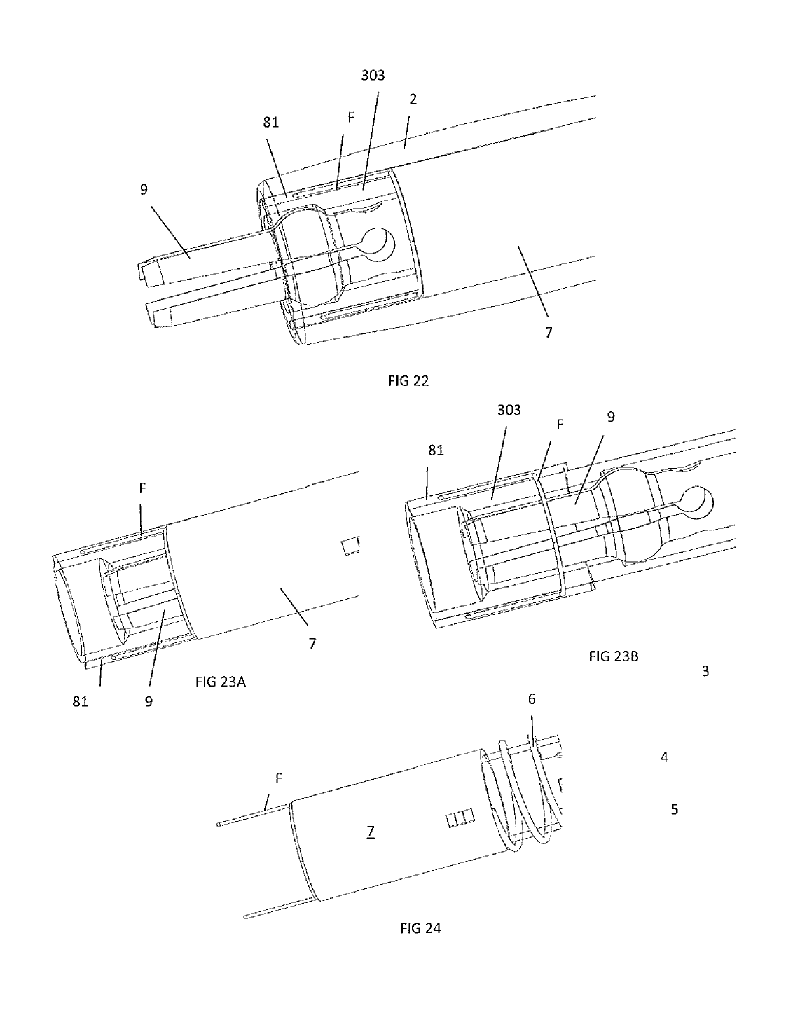

FIG. 22 clearly shows how the cutter 9 and dispenser reduced inner diameter or tapered zone 303 interact. The fastener F is located on top of the travel surface 81 waiting for being pushed by the hammer 7. The housing 2 is surrounds and keeps the fastener F over the travel surface 81. FIG. 23A through 23B is directed to the decompress stage wherein the cutter 9 is retracted inside the sutureless dispenser 3. At this stage the cutter 9 is basically fixed inside the sutureless dispenser 3. Further, FIG. 24 represents the suturing stage wherein the fastener F is pushed towards the patient.

FIG. 25A through 25B discloses the sutureless fastener member F. The sutureless fastener member F or biopsy closure fastener member comprises two prongs FA connected by a fastener body FC serving as a bridge between the prongs. The fastener body FC has resilient properties, wherein the two-pronged FA fastener may expand to a distance bigger than the diameter of the wound created by the cylindrical cutter, as shown in FIG. 25A. The fastener body material is selected from a group or resilient plastic material, resilient ceramic plastic, resilient metal material or any combination. The resilient material selected has to be a no-toxic resilient material.

The two-pronged FA are intended to be inserted at opposite sides of the wound site into the patient's skin. The sutureless fastener member F is stretched in order to be inserted into the patient's skin at opposite sides of the wound site. The stretching action is assisted by the reduce protrusion section 83 which keeps the two-pronged fasteners 4b separated enough to have a distance bigger than the diameter of the wound created by the cutter 9. After the sutureless fastener member 4 is inserted into the patient's skin the resilient properties of the sutureless fastener member F acts on the wound site. Since no rigid body is retraining the fastener member F stretched the fastener member F returns to its original form consequently making the opposite sides of the wound to come together. The resilient properties of the sutureless member F close the wound without the need of additional instruments.

While the invention has been described as having a preferred design, it is understood that many changes, modifications, variations and other uses and applications of the subject invention will, however, become apparent to those skilled in the art without materially departing from the novel teachings and advantages of this invention after considering this specification together with the accompanying drawings. For example, three stages where described however more stages can be included. For instance, if the first gear 50 is pressed the cutter shaft can returned to the load position and the cutter 9 come out of the housing 2. The action should me comparable, but not necessarily similar, to an in/out movement of a ballpoint movement. By doing this the tissue removed from the patient can be easily removed from the inner surface of the cutter 9. Accordingly, all such changes, modifications, variations and other uses and applications which do not depart from the spirit and scope of the invention are deemed to be covered by this invention as defined in the following claims and their legal equivalents. In the claims, means-plus-function clauses, if any, are intended to cover the structures described herein as performing the recited function and not only structural equivalents but also equivalent structures.

All of the patents, patent applications, and publications recited herein, and in the Declaration attached hereto, if any, are hereby incorporated by reference as if set forth in their entirety herein. All, or substantially all, the components disclosed in such patents may be used in the embodiments of the present invention, as well as equivalents thereof. The details in the patents, patent applications, and publications incorporated by reference herein may be considered to be incorporable at applicant's option, into the claims during prosecution as further limitations in the claims to patentable distinguish any amended claims from any applied prior art.

1. A biopsy punch device comprising:
a housing comprising first end and a second end, a cutter shaft assembly comprising an elongated body with a upper shaft end and a lower shaft end, a cutter and a dispenser trigger;
an sutureless dispenser actuator comprising a suture-less fastener member, a dispenser sleeve and an actuator mechanism, wherein said actuator mechanism surrounds said dispenser sleeve; and
wherein said dispenser trigger is surrounded by said sutureless dispenser actuator.

2. A biopsy punch device as in claim 1, wherein said actuator mechanism comprises a hammer and a resilient material, and wherein said hammer is a hollow structure mechanically connected to a resilient material.

3. A biopsy punch device as in claim 1, wherein said hammer comprises a hollow structure with a fastener contacting area, a sectional area, a solid hammer area and a recess.

4. A biopsy punch device as in claim 1, wherein said cutter comprises plurality of blades, a middle cutter body, tapered gap, a cutter base and fixing means; and wherein said sutureless dispenser actuator comprises a reduced inner surface, wherein said inner surface diameter is smaller than the middle cutter body.

5. A biopsy punch device as in claim 1, wherein said suture-less fastener member comprises a fastener body a first prong and second prong, wherein said fastener body comprises a fastener distal end and a fastener proximal end, wherein the first prong extend perpendicular to the fastener body and is located at the fastener distal end and wherein the second prong extend perpendicular to the fastener body and is located at the fastener proximal end.

6. A biopsy punch device as in claim 4, wherein the fastener body in made from material selected from resilient plastic material, resilient ceramic material or resilient metal material.

7. A biopsy punch device as in claim 1, wherein said cutter shaft assembly comprises:
a second resilient material, a gear assembly and a rotational pivot; wherein said gear assembly assists to retract and extend the upper shaft end.
